(19) United States
(12) Patent Application Publication
(10) Pub. No.: US 2012/0245048 A1
(43) Pub. Date: Sep. 27, 2012

(54) CELLULAR RESPONSE ASSAY FOR BIOFLUID BIOMARKER DISCOVERY AND DETECTION

(75) Inventors: Jennifer Joy SMITH, Seattle, WA (US); John David AITCHISON, Seattle, WA (US); Leslie Rae MILLER, Seattle, WA (US)

(73) Assignee: Institute For Systems Biology, Seattle, WA (US)

(21) Appl. No.: 13/429,143

(22) Filed: Mar. 23, 2012

Related U.S. Application Data
(60) Provisional application No. 61/467,898, filed on Mar. 25, 2011.

(51) Int. Cl. C40B 30/04; C12Q 1/02

(52) U.S. Cl. 506/9; 435/29

(57) ABSTRACT

The invention provides a method to detect an abnormal condition or disease state by assessing a characteristic response pattern of responder cells to indicators contained in a biological fluid of a subject. A characteristic disease-associated pattern can be obtained by determining the response pattern of responder cells to that of bodily fluids or fractions thereof from subjects known to exhibit a particular abnormal condition optionally comparing said pattern to such pattern elicited by fluids or fractions from normal subjects and identifying elements that differ. The identified elements constitute a characteristic or differential pattern which can then be used to identify the presence or stage of a disease in a test subject.

### CROSS-REFERENCE TO RELATED APPLICATION

This application claims priority from U.S. provisional application 61/467,898 filed 25 Mar. 2011. The contents of this document are incorporated herein by reference.

### TECHNICAL FIELD

The invention is in the field of diagnosis and measurement of abnormal conditions in a vertebrate subject. More precisely, it concerns using responder cells to detect indicators of the condition of a subject which indicators are present in the biological fluids of said subject.

### BACKGROUND ART

Many approaches have been used to identify markers, in particular for disease conditions. For example, expression profiles of cancer cells, as opposed to normal cells, have been used as indicators both of the presence of cancer and the stage to which it has advanced. Molecular markers such as creatine kinase MB (CK-MB) and troponin in serum have been used as indicators of myocardial infarction. Prostate-specific antigen (PSA) has been widely used as a marker for the presence of prostate cancer. Using combinations of such individual markers has also been suggested.

Levels of previously known markers of Alzheimer's disease have been measured in neuronal cells exposed to serum from elderly Alzheimer's patients, or serum from patients not afflicted with the disease. Although the markers were observed in response to disease serum, but not serum from young adults, they were not specific to Alzheimer's as they were also observed in response to serum from elderly spouses not afflicted by the disease. Neuronal viability and somal area were also assessed. Brewer, G. J., et al., J. Neurosci. Res. (1992) 33:355-359. In other studies, exposure of cultured motor neurons to serum or cerebral spinal fluid (CSF) from patients with ALS has been shown to result in increased neuronal death (Roisen, F. J., et al., Muscle Nerve (1984) 5:48-53; Couratier, P., et al., Lancet (1993) 134:265-268) and reduce abundance of ion channels (Gunasekaran, R., et al., Brain Res. (2009) 1255:170-179). In addition, conditioned medium from hepatocellular carcinoma cells was able to activate hepatic stellate cells eliciting morphological changes as well as elevation of actin expression, beta platelet derived growth factor receptor mRNA and matrix metalloprotease-2 expression. Faouzi, S., et al., Lab. Invest. (1999) 79:485-493.

The present inventors recognize that it is unlikely that any particular condition to be assessed will be associated only with a single marker that can conveniently be assayed in the biological fluid of a subject. Patterns of such markers can also be employed to enhance the accuracy of any diagnostic assessment. The complex expression patterns obtained in cellular assays are generally associated with determining the condition of the cell itself, for example, whether it is malignant or not. To applicants' knowledge, however, the complex responses of cells to multiple external factors carried, for example, in the circulation of a subject to be tested has not been employed with respect to assessing conditions associated with these unknown markers. The present invention takes advantage of the complex responses measurable in cells and thus uses responder cells as indicators of the presence or absence of any pattern of markers associated with an abnormal condition in the subject.

### DISCLOSURE OF THE INVENTION

The invention is grounded in the ability of cells to translate a complex signal or pattern, even when the nature of this signal or pattern is not known, into a measurable complex response. This is achieved by using responder cells as detectors such that a complex pattern associated with the responder cell provides the readout of the assay. Since responder cells are themselves complex systems, they provide a multiplicity of parameters that can be measured substantially simultaneously to provide a detectable pattern.

One application of this concept is to employ responder cells to assess complex changes in biological fluid of a subject which occur when the condition of the subject deviates from normal. The presence of cancer, for example, or Alzheimer's disease, or heart disease will result in a change in the contents of bodily fluids, for example, blood or cerebrospinal fluid (CSF). When these fluids are contacted with responder cells, the cells themselves exhibit a response that constitutes a complex pattern.

Thus, in one aspect, the invention is directed to a method to determine a differential response pattern characteristic of an abnormal condition or a disease stage in a subject which method comprises

a) contacting a first sample of a culture of responder cells with a biological fluid or fraction thereof of a subject known to have said abnormal condition or disease stage and determining a first response pattern of said cells to said fluid;

b) contacting a second sample of said culture of responder cells with the bodily fluid or fraction thereof of a normal subject or cells that have not been contacted with bodily fluid and determining a second response pattern of said responder cells to said fluid;

c) comparing the response pattern in a) with the response pattern in b); and

d) identifying elements or parameters of the response pattern in a) that differ from corresponding elements or parameters in b) as representing a third, differential, response pattern characteristic of said abnormal condition.

In another aspect, the invention is directed to a method to detect an abnormal condition or disease stage in a subject, by determining whether the subject has a differential response pattern characteristic of the abnormal condition or disease stage. This is done by contacting a biological fluid or fraction thereof of said subject with responder cells and determining a response pattern of said cells. The response pattern is then compared to those of control cells which have not been contacted with said biological fluid, or that are contacted with the corresponding biological fluid of a normal control subject, or to a standard normal response pattern compiled from other subjects which may be accessed in a database. This “normalized” differential response pattern can be compared to the differential response pattern determined as described above.

It is also possible directly to compare the profile obtained when a culture is exposed to a fluid of one or more subjects with a known condition or with a particular stage of said condition with the profile obtained by subjecting a second culture of cells to fluids or fractions from a subject with an unknown condition or stage. Similar profiles indicate the correspondence of the condition or stage of the test subject with that obtained from the subject who is afflicted with the known condition or stage.

In some embodiments, the response patterns are the transcriptome, and/or proteome and/or the secretion profile and/or metabolome of said cells.

### MODES OF CARRYING OUT THE INVENTION

The invention employs responder cells that will exhibit complex measurable alterations as a result of exposure to stimuli associated with a condition to be assessed. In effect, the invention method translates complex variations in the composition of fluid samples into a complex response that is reproducible and indicative of the composition pattern in the fluid.

The results of culturing the responder cells will be a pattern of a number or parameters or elements that constitutes the fingerprint of the response. Thus, the collection of individual elements or parameters measured in the responder cells or their secretions will be referred to as a “pattern” or “fingerprint” or “profile.” In this context, “parameters” and “elements” are used interchangeably.

The effectors of the response pattern will be those components of the biological fluid or fraction thereof containing them that corresponds to a particular disease, condition, or stage of progression of a disease or condition. Because these components are indicators of the disease, condition or stage, they are referred to as “indicators.” Their identity is not, and need not be, apparent from the resultant pattern.

In general, the number of parameters in the profile should be at least 3, preferably more. The number of parameters may be 3-50 or more including all integer numbers between 3 and 50.

More than one type of responder cell can be used to create the pattern where these are grown either in the same culture or in different cultures. Thus, 2, 3 or even 4 or more, such as 5-10, types of responder cells could be employed. Some may be of the type related to the condition and others unrelated to obtain patterns of complexity. Further, as the number of responder cells used in the assay is increased, it may be possible to decrease the number of parameters in the pattern for each cell. For example, four responder cells measuring a single parameter in each, such as the expression level of a single gene, could substitute for measuring the expression levels of four or five different genes in the same responder cell.

When a range such as those above is set forth in the present application, whose components are inherently integers, each of these integers is included in the specification as if specifically set forth. Further, the articles “a” and “an” are intended to mean one or more than one unless otherwise specifically indicated otherwise. “Bodily” and “biological” fluid are used interchangeably.

Means to measure multiplicities of elements or parameters—i.e., components in the resulting pattern from the response cells either internally to cells or components that are secreted by them or both are known in the art. For example, commercially available platforms can measure expression levels of hundreds or thousands of genes typically as a transcriptome—i.e., levels of mRNA present in the cell. Less developed, but nevertheless useful, are means to measure the proteome—i.e., levels of multiple proteins that are produced in the cell. Methods are also available to measure secretion profiles wherein cells may secrete multiplicities of materials into the environment. Other parameters that may be measured are the levels of various small molecules in the cells—i.e., the metabolome. Other criteria are behaviors of the cells themselves such as proliferation, changes in morphology and the like.

More complex measurements can also be obtained by measuring components of cellular regulation such as microRNA and variations in RNA splicing. Also, the compositions of molecular complexes containing protein and/or DNA can be monitored on a large scale through analyzing components of protein or chromatin immunoprecipitations.

Elements of each of these types of measurements can also be combined to obtain a complex and meaningful readout for the assays.

Further, it is not necessary that the parameters that are members of the profile be those which are known to characterize the disease; indeed, it is typically the case that the measured parameters will be different from the disease indicators. Thus, the utility of the methods of the present invention does not depend on any prior knowledge of what parameters should be measured, or what the indicators are.

In general, the test substrates are biological fluids. As used herein, “biological fluid” includes not only the fluid itself, but if desired a fraction thereof may be tested. For example, if the biological fluid is plasma or serum, it may be useful to remove albumin in order to provide a cleaner test substrate. Thus, “biological fluid” should be understood to include fractions of the fluid as well as pretreated forms thereof. Any biological fluid is appropriate as a test substrate. In addition to serum, plasma, or cerebrospinal fluid or fractions thereof, other fluids that might be tested are semen, urine, saliva and bile.

The subjects from which the biological fluids are obtained may be mammals, including primates, such as humans and laboratory models such as rabbits, rats and mice, as well as livestock such as sheep, goats, horses, cattle, and pigs and companion animals such as dogs and cats. The subjects may also be birds or fish. The responder cells may also be derived from these species and need not be of the same species as the subject. The cells need not be vertebrate cells but can derive from eukaryotes, prokaryotes or archaea. The methods of the invention may be particularly useful in combination with model systems for disease, and in testing the effects of various therapeutic protocols thereon.

In one aspect of the invention method, biological fluid from a subject or subjects with a known abnormal condition may be used to establish a baseline pattern of alteration in the responder cell components. It is not necessary to know in what manner the components of the biological fluid are altered by the abnormal condition as the pattern obtained from the responder cells is diagnostic therefor. In one embodiment, the pattern of components of the responder cells in the abnormal condition is compared with the pattern of components in the responder cells when in the presence of or having been incubated with normal biological fluid that does not exhibit the abnormal condition or that are incubated in the absence of biological fluid. The differential pattern exhibited by the responder cells, then, can be used as a standard for comparison to similar results from test samples. Thus, a differential pattern can be established by identifying those elements of a response pattern exhibited as a result of contact with bodily fluids representing an abnormal condition from those elements in patterns established by contacting fluids derived from subjects that are normal, and creating a pattern that includes these elements or that are not contacted by such fluids. This embodiment has the advantage of leaving out parameters that are not indicative of the disease itself. However, as further discussed below, it is possible simply to directly compare the pattern of parameters obtained from a subject with known abnormal conditions or stage of disease with that of a test subject.

Thus, a “differential pattern” refers to a pattern obtained by comparing the pattern from a subject with a known condition or stage of condition with a normalizing pattern or a pattern obtained from a test subject with a normalizing pattern. The normalizing pattern is typically a pattern obtained when the responder cells are contacted with fluids or fractions from one or more normal subjects. Other types of normalizing patterns could also be used, such as a pattern obtained when the cells are simply cultured in the absence of any extraneous fluids other than typical culture media. In both cases, the differences can be evaluated statistically depending on the number of subjects included in any of these groups. Thus, if sufficient numbers of independent patterns are used, statistically significant differences can be evaluated, and if desired, used as a criterion for including a specific parameter in the final pattern or profile. In some cases, only a single subject may be used to create a standard for abnormal conditions or disease stage and a single normal control may be used.

While theoretically any cells could be used as responder cells, the information obtainable from the assay may be improved if the responder cells are selected from those cells that are expected to show a response to whatever changed compositions are associated with an abnormal condition. For example, for conditions that are associated with abnormalities in the neuronal system, neuronal cells may be used on responders. For assessment of abnormal conditions that exhibit their biological effects on the hematopoietic system, hematopoietic cells are favored. This nexus, however, is not necessary, and any cell that provides a sufficiently nuanced pattern in response to an abnormal condition could be used.

In general, the differential response pattern can be established by comparing responses obtained from fluids obtained from abnormal subjects with those obtained from fluids of normal subjects. The responses can be compared by identifying individual transcripts that are significantly differentially expressed between the two responses, or by generating and testing a more complex disease classification model using approaches such as support vector machines or random forest algorithms (Statnikov, A., et al., BMC Bioinformatics (2008) 9:319). Such algorithms can identify diagnostic signatures composed of sets of candidate transcripts and disease classification decision rules (which can be based on more complex aspects of the data such as the relative intensities of two different transcripts in the same sample).

The analysis can be expanded to obtain a longitudinal or cross sectional set of disease signatures, by obtaining complex multicomponent readouts from responder cells (e.g., gene expression microarrays) after exposure to sera from normal or diseased subjects taken at various stages of disease progression. In longitudinal studies, a single subject at various disease stages is assessed, whereas in cross-sectional diagnoses, multiple subjects at various disease stages can be used as subjects.

One illustrative embodiment to handling data showing disease progression is to construct a differential response pattern made up of log₂ expression ratios (disease serum exposure/normal serum exposure) obtained of responder-cell genes in the cultured cells that are good indicators of disease progression. Expression values of various genes for disease subjects at each stage of progression are evaluated relative to matched normal subjects (e.g., subjects can be matched with respect to genetics, age and/or environment). This sets a standard pattern to which expression level data obtained similarly with respect to fluids of a test subject can be compared. If a large number of genes make up the signature, it is possible, if desired, to cluster genes in some way; for example, genes with similar response profiles (data in rows) could be clustered. Categorizing genes in this way enables recognition of higher-level disease signatures such as response characteristic of a cellular process rather than expression level of an individual gene.

To apply the results obtained above, the responder cell assay is performed with serum from the subject and disease state is assessed by mapping to the longitudinal progression pattern. This is done by obtaining readouts from responder cells after exposure to query serum of a test subject and control serum using the same experimental conditions that were used to generate the longitudinal (progression) data. Here the control is non-disease serum from a genetically matched subject of the same age, but for other disease applications, it could be serum taken from the subject itself before disease onset.

Alternatively, the pattern obtained from various subjects representing the stages of disease progression could be compared directly with the expression pattern obtained from a test subject to match directly the expression pattern in the respondent cells from fluids in the test subject with the corresponding pattern in the known disease stage.

Assessing disease stage can also be useful in evaluating treatment. The expression pattern of responder cells characteristic of a particular stage of a disease, whether or not normalized to that of normal subjects can be compared to the pattern obtained from a test subject before and after treatment, again, either directly or where both patterns have been normalized to normal subjects. Effectiveness of the treatment is reflected in finding that the pattern in the test subject represents an earlier stage of progression than was exhibited before treatment. Thus, if before treatment the subject exhibits the pattern characteristic of stage 5, successful treatment would be indicated if the pattern after treatment is representative of disease stage prior to 5, such as disease stage 3.

Even with respect to conditions that are not directly specific to a particular type of cell, such as malignancies, secretions from malignant cells to, for example, the bloodstream, permit the blood to be used as a test sample in which case the responder cells could be those of normal tissue associated with the cancer itself.

None of these pairings are required; the complexity of metabolism makes it possible to use virtually any cells as responder cells regardless of whether they are primary targets of the altered composition of the biological fluid.

It is also possible to identify components of a biological fluid that effect the complex changes in the responder cells if it is the case that only one or two or a few markers are responsible. The biological fluid can be fractionated and each fraction evaluated separately with respect to changes in the responder cell pattern. On the other hand, if it is a complex mixture of altered components in the biological fluid that is responsible for the pattern alterations in the responder cell, the task becomes more complex.

The following examples are included to illustrate but not to limit the invention.

### EXAMPLE 1

### Use of Motor Neurons to Detect Amyotrophic Lateral Sclerosis (ALS) Susceptibility in Serum

ALS is a human neurodegenerative disease caused by progressive degeneration of motor neurons that control voluntary muscle movement. The molecular mechanisms of disease progression are not known, but evidence suggests that ubiquitous activation of the subcellular misfolded protein response cascades to a dysfunctional interaction between motor neurons and supporting astrocytes, resulting in neuronal death. Most ALS cases are sporadic, while 10% of cases are inherited. Dominant mutations of the SOD1 gene are among the genetic defects that have been reported to cause ALS. A transgenic mouse model of ALS has been generated, which is hemizygous for G93A mutant form of human SOD1.

Although the unfolded protein response is detectable in motor neurons of the mice by about 7 weeks of age, the mice have only minor symptoms of disease until about 11 weeks of age when body weight starts to significantly decline. Symptoms progress to neuromuscular dysfunction at about 13 weeks, followed by paralysis by 19-20 weeks.

In this example, the ALS signature in blood serum of mice developing ALS was determined using motor neurons as detector cells. Motor neurons have been shown to be targeted by the disease in a non cell autonomous manner (Nagai, M, et al., Nature Neuroscience (2007) 10:615-622), and therefore may be responsive to disease-specific signatures in serum.

Disease serum was taken from 5 transgenic ALS susceptible mice (SOD1; G93A) at 9 weeks of age and control serum was taken from 5 non-carrier mice of the same age from the same colony.

Spinal motor neurons (MNs) were derived from HGB3 embryonic stem cells expressing a fluorescently labeled motor neuron marker (HB9-eGFP) by a method previously described (Wichterle, H., et al., Cell (2002) 110:385-397) as described below. Unless otherwise specified, growth of ES cells was in differentiation medium (consisting of equal parts Advanced™ DMEM/F12 (Invitrogen) and Neurobasal™ medium (Invitrogen) supplemented with penicillin/streptomycin, 2 mM L-Glutamine, 0.1 mM 2-mercaptoethanol, and 10% KnockOut™ serum replacement (Invitrogen)). ES cells were plated at ˜10⁵ cells per mL and grown in aggregate culture for 2 days to form embryoid bodies (EBs) in a 10 cm² dish. EBs were split 1:4 into four 10 cm² dishes and exposed to 1 μM each retinoic acid and sonic hedgehog agonist (Hh-Ag1.3, Curis, Inc.) for two days, to caudalize spinal character and ventralize into MN progenitors, respectively. Medium was changed and EBs were grown for an additional 3 days in differentiation medium to generate MNs. Two dishes of EBs were pooled, washed with PBS and resuspended in 1 mL of differentiation medium. 100 μL it of these EBs were inoculated in each of 10 wells of a 3.8 cm² 12-well dish. EBs were incubated for 24 h in 2 mL differentiation medium containing either 5% serum from 9 week-old ALS susceptible mice or 5% serum from normal mice. Each experiment (disease or control) was done five times with serum from five different mice.

RNA was isolated using TRIzol® reagent, and cDNA was synthesized from polyA RNA, labeled and hybridized to Affymetrix GeneChip® mouse exon arrays according to manufacturer's recommendations.

Probe intensities for ten experiments (five replicates each of control and disease serum) were normalized together and data from probes representing a continuous stretch of putatively transcribed genomic sequence were merged into probe sets (using RMA algorithm of the Affymetrix Expression Console software). Two filters were applied to exclude probe sets that did not meet the criteria below: 1. Probe sets map to the genome and thus levels are annotated as “core”, “full”, “free” or “extended” by Affymetrix. 2. Probe sets have high confidence of detection over background in at least 5 of the 10 experiments (P<0.001 determined using the DABG algorithm of the software). After application of these two filters, the data set consisted of 135,181 probe sets.

Probe-level expression values were analyzed for significant differential expression between cells exposed to control serum and those exposed to disease serum using Significance Analysis for Microarrays (SAM) of MeV component of TM4 microarray software (by running a two-class paired analysis using default parameters and the 32 possible unique permutations of the data to calculate the statistic). This analysis generated an ALS disease signature consisting of 441 probe sets that significantly increased in expression in response to disease serum compared to normal serum with q-values and false discovery rates <15%.

The differential response pattern need not reflect the underlying biology of disease progression in order to be used as a classifier of disease state; however significant overrepresentation of disease relevant genes in the signature supports the contention that the measured cellular response is specific to the disease rather than background or noise. The disease signature of 441 exons contained four probe sets in three genes that are causatively associated with ALS including SETX (Chen, Y -Z., et al., Am J. Hum. Genet. (2004) 76:1128-1135), TARDBP (Sreedharan, J., et al., Science (2008) 319:1668-16672; Yokoseki, A., et al., Ann. Neurol. (2008) 63:538-542; Rutherford, N. J., et al., PLoS Genetics (2008) 4:e1000193), and VCP (Johnson, B. S., et al., Neuron (2010) 68:857-864). Nine genes have been identified to have mutations linked to ALS (Strong, M. J., J. Neurological Sciences (2010) 288:1-12; Johnson, B. S., et al., Neuron (2010) 68:857-864), and three of them are in the disease signature (representing 400 of the 30,000-40,000 total genes). This overrepresentation of disease genes in the signature is significant with HGD p-value of ˜1×10⁻⁶).

This cell-mediated disease signature could be used to classify the disease states of test subjects from their serum. A possible approach is to generate more replicates of the data with known disease and normal samples, and apply support vector machines or random forest algorithms (Statnikov, A., et al., BMC Bioinformatics (2008) 9:319) to build and test a disease classification model. The models can be tested using cross-validation and then validated using independent test sets of mice. In this case, test subjects would be analyzed by measuring the global expression response of responder cells to serum as was done to build the model, but in other applications, test subject analysis might involve measuring only the response of transcripts that comprise the diagnostic signature using targeted approaches for quantification of selected transcripts.

### EXAMPLE 2

### Use of Endothelial Progenitor Cells (EPCs) and Other Vascular Progenitor Cells as Biosensors of Malignant Tumors, Proliferative Retinopathy and Ischemia

Angiogenesis or blood vessel formation is a fundamental step in malignant tumor formation and is also associated with other health concerns including proliferative retinopathy associated with diabetes, and ischemia associated with stroke and heart disease. It is mediated by mobilization and recruitment of bone marrow-derived endothelial precursor cells (including endothelial progenitor cells (EPCs), and hematopoietic stem and progenitor cells). Recruitment of these cells to the target location is mediated by signaling molecules in the serum (including cytokines, angiogenic factors, platelet-derived growth factors, and as of yet uncharacterized factors), which likely comprise organ-specific signatures. In addition, it is thought that upon reaching the target tissue, differentiation of the progenitor cells is influenced by specific signaling molecules including local extracellular matrix components. Therefore a potential biosensor assay for tumor development and other cell proliferative conditions includes the use of detector cells that are EPCs and other vascular progenitor cells (which can be isolated from bone marrow, or derived from embryonic stem cells), and the use of blood serum as the biofluid. We anticipate that this assay will be particularly valuable for detection and characterization of malignant tumors as they have been linked to local secretion of destructive proteases (both matrix metalloproteases and serine and threonine proteases) that break down extracellular matrix components releasing locally confined growth factors and polysaccharides that regulate cell behavior into the blood stream.

### EXAMPLE 3

### Use of Stromal Cells and Other Cells Involved in Desmoplastic Reaction as Biosensors of Various Malignant Tumors

Desmoplastic reaction is the growth of fibrous or connective tissue in response to neoplasm, resulting in the formation of dense fibrosis around a tumor. This is usually associated specifically with malignant neoplasms. The reaction involves stromal cells that are considerably diverse depending on environmental cues including heterologous interactions with other cell types in the stroma and differential regulation by tumor cells. For example, tumor cells signal to various stromal cells including fibroblasts, which are transformed into myofibroblasts that produce collagen and other ECM proteins (Desmouliere, et al., Int. J. Dev. Biol. (2004) 48:509-517), inflammatory cells that have a role in tumor immunity (Coussens, et al., Nature (2002) 420:860-887), and endothelial cells that have roles in neoangiogenesis and tumor progression (Carmeliet, et al., Nature (2000) 407:249-257). Therefore, potential cell-based assays to detect malignant neoplasm and possibly the evolutionary stage and location of the tumor involve the use of stromal cells involved in desmoplastic reaction (either in isolation or in a heterologous mixture to leverage cell-cell communications to amplify the signal). For example, a potential biosensor of hepatocellular carcinoma is a mixture of involved stromal cells, including hepatic stellate cells (fibroblasts), sinusoidal endothelial cells and inflammatory Kupffer cells (reviewed in Desmouliere, et al., supra). In this heterologous stromal cell detection system, serum from diseased patients is expected to transform fibroblasts into myofibroblasts among other responses that make up a specific signature of hepatocellular carcinoma that is different from other desmoplastic responses to other tumor types and wound repair. In breast tissue, the fibrous tissue is fatty and is presumably derived from adipose cells. Therefore, a potential biosensor assay for breast cancer development includes the use of stromal cells including adipocytes as responder cells to detect molecular breast cancer signature in blood serum. Supporting these approaches, exposure of adipose cells to media conditioned by different human breast cancer cell lines induces reversion of adipocytes to fibroblastic cells. In addition, exposure of hepatic stellate cells to media conditioned by rat hepatocellular carcinoma cell lines, increased cell proliferation, alpha-smooth muscle actin expression and gelatinase A secretion. Faouzi, et al., Lab. Invest. (1999) 79:485-493.

### EXAMPLE 4

### Use of Neurons as Biosensors of Neurodegenerative Diseases Including Alzheimer's Disease, Amyotrophic Lateral Sclerosis and Parkinson's Disease

Several neurodegenerative diseases result in the selective destruction a specific neuronal population. The cause of neuronal loss is not known, but in some cases it has been linked to altered environment of the neuron including signaling from other cells, rather than the neurons themselves. Additionally, it has been shown that biomarkers of disease in cerebral spinal fluid can also be found in the blood. For these reasons, neurons that are targeted by neurodegenerative diseases are potential biosensors of disease-specific signatures in blood serum and cerebral spinal fluid. Specifically motor neurons, glutamatergic and cholinergic neurons, and dopamine neurons are potential biosensors of amyotrophic lateral sclerosis, Alzheimer's disease, and Parkinson's disease, respectively. These neurons can be derived from embryonic stem cells or neuroblastoma cell lines. Hill and Robertson, Brain Res. Prot. (1998) 2:183-190.

1. A method to determine a response pattern characteristic of an abnormal condition or a disease stage in a subject which method comprises contacting a first sample of a culture of responder cells with a biological fluid or fraction thereof of at least one subject known to have said abnormal condition or disease stage and determining a first response pattern of said cells to said fluid.

2. The method of claim 1 which further comprises comparing the first response pattern with a second normalizing response pattern which has been obtained by contacting a second sample of said culture of responder cells with biological fluid or fraction thereof of one or more normal subjects or culturing said responder cells in the absence of extraneous biological fluid and determining said second response pattern of said responder cells and
identifying elements of the first response pattern that differ from corresponding elements in the second as representing a third, differential, response pattern characteristic of said abnormal condition or disease stage.

3. The method of claim 1 wherein the response patterns are transcriptome and/or proteome and/or metabolome and/or secretion profiles of the responder cells.

4. The method of claim 1 wherein the method employs at least two types of responder cells.

5. The method of claim 1 wherein the response pattern characteristic of an abnormal condition or disease stage contains at least five elements.

6. The method of claim 2 wherein the response patterns are transcriptome and/or proteome and/or metabolome and/or secretion profiles of the responder cells.

7. The method of claim 2 wherein the method employs at least two types of responder cells.

8. The method of claim 2 wherein the differential response pattern characteristic of an abnormal condition or disease stage contains at least five elements.

9. The method of claim 1 wherein the subject is human.

10. The method of claim 1 wherein the subject is a laboratory model of said condition.

11. A method to detect an abnormal condition or disease stage in a test subject, which method comprises determining whether said test subject is associated with a response pattern characteristic of said condition or stage by a method comprising contacting a biological fluid or fraction thereof of said test subject with a culture of responder cells and determining a response pattern of said cells to the fluid or fraction, and
comparing the response pattern of said test subject to the response pattern determined by the method of claim 1 as characteristic of the abnormal condition or disease stage
whereby similarity of the pattern obtained from the test subject to the pattern characteristic of the abnormal condition or disease stage detects the abnormal condition or disease stage in said subject.

12. The method of claim 11 wherein the pattern is a transcriptome and/or proteome and/or metabolome and/or secretion profile of said cells.

13. The method of claim 11 wherein the method employs at least two types of responder cells.

14. The method of claim 11 wherein the response pattern characteristic of an abnormal condition or disease stage contains at least five elements.

15. The method of claim 11 wherein the subject is human.

16. The method of claim 11 wherein the subject is a laboratory model of said condition.

17. A method to detect an abnormal condition or disease stage in a test subject, which method comprises determining whether said subject is associated with a differential response pattern characteristic of said condition or stage by a method comprising
a) contacting a biological fluid or fraction thereof of said subject with a culture of responder cells and determining a response pattern of said cells to the fluid,
b) comparing the response pattern of said subject in a) to a response pattern of control cells that have been contacted with biological fluid or fraction thereof of a normal subject or culturing said responder cells in the absence of extraneous biological fluid to obtain a differential response pattern associated with the subject,
c) comparing the differential pattern in b) with a characteristic, differential, pattern determined by the method of claim 2
whereby similarity of the differential pattern associated with the subject to the differential pattern characteristic of the abnormal condition or disease stage detects the same in said subject.

18. The method of claim 17 wherein the pattern is a transcriptome and/or proteome and/or metabolome and/or secretion profile of said cells.

19. The method of claim 17 wherein the method employs at least two types of responder cells.

20. The method of claim 17 wherein the differential response pattern characteristic of an abnormal condition or disease stage contains at least five elements.

21. The method of claim 17 wherein the subject is human.

22. The method of claim 17 wherein the subject is a laboratory model of a disease or condition.

23. The method of claim 11 which is performed at least two different times on a fluid or fraction from a test subject in order to determine disease progression.

24. The method of claim 11 which is performed at least two times on a fluid or fraction from a subject treated with a protocol, wherein said method is performed before and after treatment with said protocol to determine effectiveness of said protocol.

25. The method of claim 17 which is performed at least two different times on a fluid or fraction from a test subject in order to determine disease progression.

26. The method of claim 17 which is performed at least two times on a fluid or fraction from a subject treated with a protocol, wherein said method is performed before and after treatment with said protocol to determine effectiveness of said protocol.

27. The method of claim 2 wherein the subject is human.

28. The method of claim 2 wherein the subject is a laboratory model of said condition.
